(19) United States
(12) Patent Application Publication
(10) Pub. No.: US 2025/0372211 A1
(43) Pub. Date: Dec. 4, 2025

(54) APPARATUS AND METHOD OF HIGH THROUGHPUT OXIDATION SIMULATION FOR SELECTING ULTRA HIGH TEMPERATURE CERAMICS

(71) Applicant: Foundation of Soongsil University Industry Cooperation, Seoul (KR)

(72) Inventors: Jiwoong KIM, Seoul (KR); Junyoung CHOI, Seoul (KR); Hyokyeong KIM, Seoul (KR); Hayoung SON, Seoul (KR)

(21) Appl. No.: 19/226,606

(22) Filed: Jun. 3, 2025

(30) Foreign Application Priority Data
Jun. 3, 2024 (KR) 10-2024-0072597

(51) Int. Cl. G16C 20/30

(52) U.S. Cl. CPC G16C 20/30

(57) ABSTRACT

The present disclosure relates to a high throughput oxidation simulation apparatus and method for selecting ultra-high temperature ceramics. A high throughput oxidation simulation apparatus according to the present disclosure generates a model for calculating physical properties of candidate materials based on both a material intended for selecting ultra-high temperature ceramics and the type and amount of dopant elements, calculates mechanical properties of the candidate materials using the generated model, predicts oxidation rates of the candidate materials, and predicts surface oxide compositions of the candidate materials. By selecting ultra-high temperature ceramic materials based on the mechanical properties, oxidation rates, and surface oxide compositions of the candidate materials, time and cost required for selecting ultra-high temperature ceramics can be reduced.

### CROSS-REFERENCE TO RELATED APPLICATION

The present application claims priority under 35 U.S.C. § 119 (a) to Korean patent application number 10-2024-0072597 filed on Jun. 3, 2024, in the Korean Intellectual Property Office, the entire disclosure of which is incorporated by reference herein.

### BACKGROUND

### 1. Technical Field

The present disclosure relates to simulation technology for material design, and more particularly, to simulation technology for selecting optimal compositions for ultra-high temperature ceramics.

### 2. Related Art

Materials such as ultra-high temperature ceramics are used in extreme environments, including defense and aerospace applications, and thus must possess excellent heat resistance and oxidation resistance without degradation at ultra-high temperatures of 2,000° C. or higher.

Accordingly, to develop and test such materials, it is essential to reproduce extreme environments such as ultra-high temperatures to identify changes in material properties. However, directly measuring the oxidation of a specimen under ultra-high temperature conditions is practically impossible.

To address this, conventional technologies have employed arc-jet methods to simulate ultra-high temperature environments. However, such arc-jet-based methods only allow observation of the material's state after the high-temperature oxidation simulation is completed, making it nearly impossible to identify oxidation reaction mechanisms occurring during the oxidation process. In addition, the implementation of an evaluation environment to simulate such an ultra-high temperature oxidation condition is extremely limited and incurs substantial costs, which presents a significant barrier to easy access.

After extensive research to solve the above problems of the conventional technology, the inventors of the present disclosure have completed the present disclosure, which enables efficient selection of optimal compositions based on the essential requirements of ultra-high temperature ceramics.

### SUMMARY

To solve the problems of the related arts described above, the present disclosure is directed to providing an apparatus and method capable of predicting the oxidation mechanism of a material in a theoretical environment rather than through ultra-high temperature simulation experiments.

The present disclosure is also directed to providing a method for predicting the high-temperature oxidation mechanism of a material under development, thereby enabling more efficient planning of experiments and reducing empirical trial and error.

The technical problems addressed by the present disclosure are not limited to those mentioned above and other problems will be readily apparent to those of ordinary skill in the art from the following description.

To solve the above problems, a high throughput oxidation simulation apparatus for selecting ultra-high temperature ceramics according to a preferred embodiment of the present disclosure may include a memory storing one or more instructions; and a processor configured to execute the one or more instructions stored in the memory, and the processor may be configured to generate a model for calculating physical properties of candidate materials based on both a material intended for selecting ultra-high temperature ceramics and the type and amount of dopant elements, calculate mechanical properties of the candidate materials using the generated model, predict oxidation rates of the candidate materials, predict surface oxide compositions of the candidate materials, and select ultra-high temperature ceramic materials based on the mechanical properties, oxidation rates, and surface oxide compositions of the candidate materials.

The processor may be configured to calculate the oxidation rates of the candidate materials through NEB (nudged elastic band) calculations.

The processor may be configured to calculate the surface oxide compositions and oxidation rates of the candidate materials through AIMD (Ab initio Molecular Dynamics) simulation.

The processor may be configured to predict the oxidation rates and surface oxide compositions of the candidate materials only when the mechanical properties of the candidate materials meet or exceed a predetermined criterion.

The processor may be configured to calculate the synthesizability of the dopant elements for the candidate materials when the mechanical properties of the candidate materials meet or exceed a predetermined criterion, and to predict the oxidation rates and surface oxide compositions of the candidate materials only when the dopant elements are determined to be synthesizable.

A high throughput oxidation simulation method according to a preferred embodiment of the present disclosure may include generating a model for calculating physical properties of candidate materials based on both a material intended for selecting ultra-high temperature ceramics and the type and amount of dopant elements; calculating mechanical properties of the candidate materials using the generated model; predicting oxidation rates of the candidate materials; predicting surface oxide compositions of the candidate materials; and selecting ultra-high temperature ceramic materials based on the mechanical properties, oxidation rates, and surface oxide compositions of the candidate materials.

The step of predicting oxidation rates may include calculating the oxidation rates of the candidate materials through NEB (nudged elastic band) calculations.

The step of predicting surface oxide compositions may include calculating the surface oxide compositions and oxidation rates of the candidate materials through AIMD (Ab initio Molecular Dynamics) simulation.

After the step of calculating mechanical properties, the oxidation rates and surface oxide compositions of the candidate materials may be predicted only when the mechanical properties of the candidate materials meet or exceed a predetermined criterion.

The method may further include, after the step of calculating mechanical properties, calculating the synthesizability of the dopant elements for the candidate materials when the mechanical properties of the candidate materials meet or exceed a predetermined criterion.

According to the present disclosure, it is possible to establish a direction for developing materials with desired properties without repeated trial and error for property prediction in ultra-high temperature environments.

In addition, by conducting ultra-high temperature experiments only on such screened materials, there is an advantage of saving time and cost for ultra-high temperature experiments.

The effects of the present invention are not limited to those mentioned above, and other effects not mentioned will be clearly understood by those of ordinary skill in the art from the following description.

### BRIEF DESCRIPTION OF THE DRAWINGS

FIG. 1 is a schematic structural diagram of a high throughput oxidation simulation apparatus for selecting ultra-high temperature ceramics according to a preferred embodiment of the present disclosure.

FIG. 2 illustrates an example of mechanical property simulation results according to a preferred embodiment of the present disclosure.

FIG. 3 illustrates an example of competing phase and formation energy calculation results according to a preferred embodiment of the present disclosure.

FIG. 4 illustrates an example of NEB (nudged elastic band) calculation results according to a preferred embodiment of the present disclosure.

FIG. 5 illustrates an example of AIMD (ab initio molecular dynamics) simulation results according to a preferred embodiment of the present disclosure.

FIG. 6 is a schematic flowchart of a high throughput oxidation simulation method for selecting ultra-high temperature ceramics according to another preferred embodiment of the present disclosure.

### DETAILED DESCRIPTION

The above-mentioned objects, means, and effects thereof of the present disclosure will become more apparent from the following detailed description in relation to the accompanying drawings, and accordingly, those skilled in the art to which the present disclosure belongs will be able to easily practice the technical idea of the present disclosure. In addition, in describing the present disclosure, when it is determined that a detailed description of a related known technology may unnecessarily obscure the subject matter of the present disclosure, the detailed description will be omitted.

The terms used in this specification are for the purpose of describing embodiments only and are not intended to limit the present disclosure. In this specification, the singular forms “a,”, “an,” and “the” also include plural forms in some cases unless otherwise specified in the context. In this specification, terms such as “include”, “comprise”, “provide” or “have” do not exclude the presence or addition of one or more other elements other than elements mentioned.

In this specification, terms such as “or” and “at least one” may represent one of the words listed together or a combination of two or more thereof. For example, “A or B” and “at least one of A and B” may include only one of A or B, or may also include both A and B.

In this specification, descriptions according to “for example”, etc. may not exactly match the information presented, such as the recited properties, variables, or values, and effects such as modifications, including tolerances, measurement errors, limits of measurement accuracy, and other commonly known factors should not limit the modes for carrying out the invention according to the various exemplary embodiments of the present disclosure.

In this specification, when an element is described as being “connected” or “linked” to another element, it will be understood that it may be directly connected or linked to the other element, but intervening elements may also be present. On the other hand, when an element is referred to as being “directly connected” or “directly linked” to another element, it will be understood that there are no intervening elements present.

In this specification, when an element is described as being “on” or “adjacent to” another element, it will be understood that it may be directly “on” or “connected to” the other element, but intervening elements may also be present. On the other hand, when an element is described as being “directly on” or “directly adjacent to” another element, it will be understood that there are no intervening elements present. Other expressions describing the relationship between the elements, for example, “between” and “directly between”, and the like can be construed similarly.

In this specification, terms such as “first” and “second” may be used to describe various elements, but, the above elements should not be limited by the terms above. In addition, the above terms should not be construed as limiting the order of each element, and may be used for the purpose of distinguishing one element from another. For example, a “first element” may be named as a “second element” and similarly, a “second element” may also be named as a “first element.”

Unless otherwise defined, all terms used in this specification may be used with meanings commonly understood by those of ordinary skill in the art to which the present disclosure belongs. In addition, terms defined in a commonly used dictionary are not interpreted ideally or excessively unless explicitly and specifically defined.

Hereinafter, preferred embodiments according to the present disclosure will be described in detail with reference to the accompanying drawings.

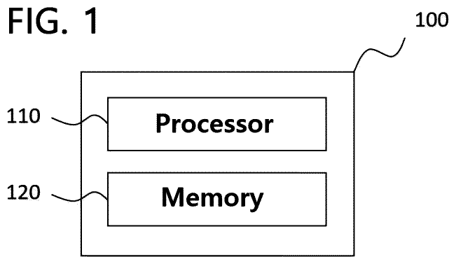

FIG. 1 is a schematic structural diagram of a high throughput oxidation simulation apparatus according to a preferred embodiment of the present disclosure.

The high throughput oxidation simulation apparatus 100 according to the present disclosure may include one or more processors 110 and a memory 120.

The memory 120 may store instructions, data structures, and program code readable by the processor 110. In some embodiments, at least the operations performed by the processor 110 may be implemented by executing instructions or code of a program stored in the memory 120.

The memory 120 may include a flash memory type, a hard disk type, a multimedia card micro type, or a card-type memory (e.g., SD or XD memory), and may include non-volatile memory such as ROM (Read-Only Memory), EEPROM (Electrically Erasable Programmable Read-Only Memory), PROM (Programmable Read-Only Memory), magnetic memory, magnetic disks, or optical disks, and volatile memory such as RAM (Random Access Memory) or SRAM (Static Random Access Memory).

The memory 120 may store one or more instructions or programs that the high throughput oxidation simulation apparatus 100 may use to perform a simulation for selecting ultra-high temperature ceramics.

The processor 110 controls the overall operations of the high throughput oxidation simulation apparatus 100. For example, the processor 110 may control the overall operation for selecting ultra-high temperature ceramics by executing one or more instructions stored in the memory 120, such that the high throughput oxidation simulation apparatus 100 generates candidate materials and performs high-temperature oxidation simulations.

The processor 110 may include, for example, at least one of a central processing unit (CPU), a microprocessor, a graphics processing unit (GPU), an application-specific integrated circuit (ASIC), a digital signal processor (DSP), a digital signal processing device (DSPD), a programmable logic device (PLD), a field programmable gate array (FPGA), an application processor, a neural processing unit (NPU), or a processor dedicated to artificial intelligence designed with a hardware architecture specialized for processing AI models, but is not limited thereto.

The processor 110 first generates a model for calculating the physical properties of candidate materials that may be used as ultra-high temperature ceramics.

The candidate materials may include, for example, pure materials and hetero elements doped thereto.

The processor 110 may generate and use a model for generating candidate materials that vary depending on the type and amount of elements doped, that is, added to the candidate materials. The generated model may automatically perform first-principles calculations (Ab initio) based on the type and amount of the added elements.

First-principles calculation (ab initio) refers to an approach that fundamentally derives governing laws based on firmly established physical principles (e.g., electron interactions) without relying on empirical data or experimental data used for fitting. In other words, it is a method of analyzing the properties of a material by calculating the interactions (forces) between electrons through their density function.

The processor 110 first calculates the mechanical properties of the candidate materials to perform high-throughput screening.

For the screening, the processor 110 may apply strain to the candidate materials to generate strained models and may calculate elastic constants through energy calculations.

The processor 110 may first determine the mechanical properties of the candidate materials for high-throughput screening.

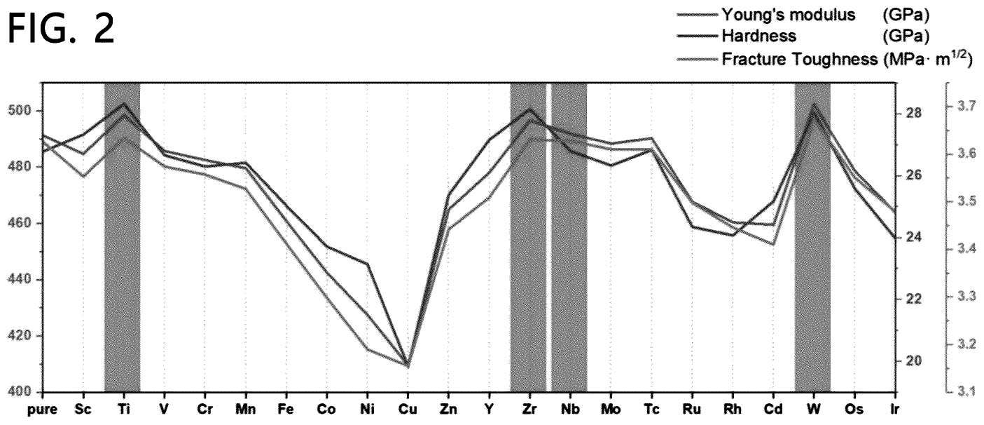

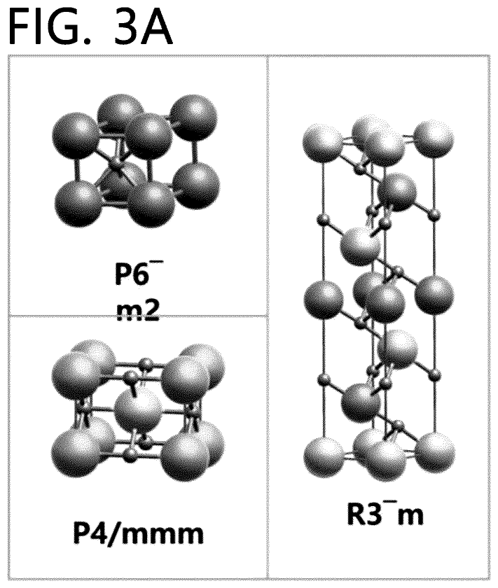

FIG. 2 illustrates an example of high-throughput screening of candidate materials based on their mechanical properties.

FIG. 2 illustrates an example in which mechanical properties—namely, Young's modulus, hardness, and fracture toughness—are calculated according to dopant elements, and dopant elements that enhance the mechanical properties compared to a pure material are selected.

By identifying dopant elements that yield superior mechanical properties compared to the undoped pure material based on the mechanical property calculation results, candidate materials can be initially screened.

Since mechanical properties are as important as oxidation characteristics at ultra-high temperatures for materials to be used as ultra-high temperature ceramics, high-throughput screening may first be performed based on mechanical properties to narrow down the number of candidate materials for subsequent oxidation simulations. Accordingly, by reducing the number of oxidation simulations, which require significantly greater computational power, the total number of simulations needed for selecting ultra-high temperature ceramics can be decreased, thereby reducing the overall time required for the simulations.

The processor 110 may first verify the synthesizability of the dopant elements for the candidate materials that have been primarily screened based on mechanical properties. This is because subsequent steps are unnecessary for dopant elements that are not synthesizable.

The synthesizability of the dopant elements is determined by calculating the doping energy of the hetero elements through formation energy calculations and comparison with competing phases.

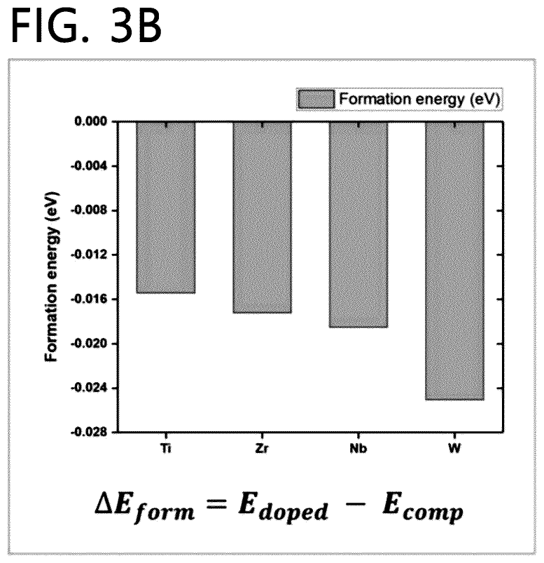

FIG. 3 illustrates an example of formation energy and competing phase comparison for verifying synthesizability.

FIG. 3A shows examples of competing phases, and FIG. 3B shows a comparison of formation energies according to dopant elements.

After the primary screening and verification of synthesizability, oxidation simulations at ultra-high temperatures are performed in earnest.

For oxidation simulation, the processor 110 first calculates the oxidation rate of the candidate materials that have passed the primary screening.

The oxidation rate of a material is determined by the diffusion rate of oxygen, which can be predicted through NEB (nudged elastic band) calculations.

NEB calculation is a simulation method used to determine the migration path and energy barrier of atoms within a molecule or solid. By searching for the most optimal path between the initial and final states, NEB calculation can estimate the energy barrier and activation energy of the reaction path. Therefore, the oxidation reaction mechanism and rate at ultra-high temperatures can be predicted using this method.

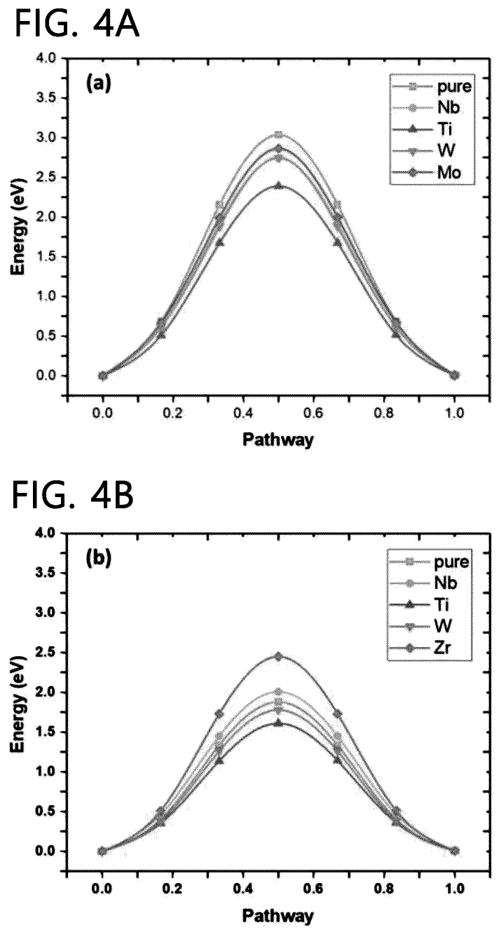

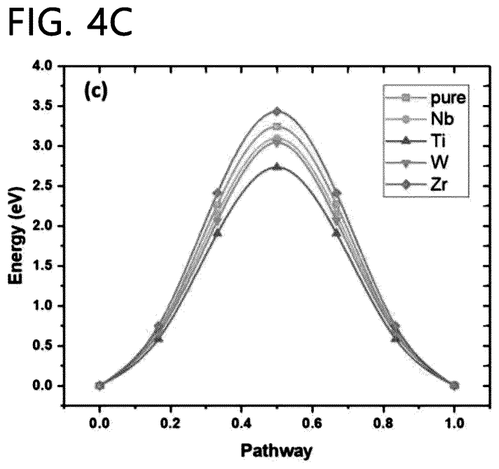

FIG. 4 illustrates the results of oxidation rate prediction using NEB calculations.

FIGS. 4A, 4B, and 4C respectively show the predicted energy barriers of oxygen for ZrC, HfC, and TaC according to different dopant elements, as calculated through NEB simulations. Through such calculations, the oxidation rates of candidate materials for each dopant element can be compared.

Finally, the processor 110 predicts the composition of surface oxides of the candidate materials at ultra-high temperatures.

AIMD (Ab initio Molecular Dynamics) simulation is used for oxide composition prediction. AIMD simulation is a method that overcomes the limitations of conventional molecular dynamics simulations by applying first-principles calculation (Ab initio).

AIMD simulation can model various physical and chemical properties of molecules by simultaneously considering both electronic motion and nuclear motion. Accordingly, the composition of oxides of the candidate materials at ultra-high temperatures can be predicted through AIMD simulation, and the oxidation reaction rate can be estimated based on the predicted composition.

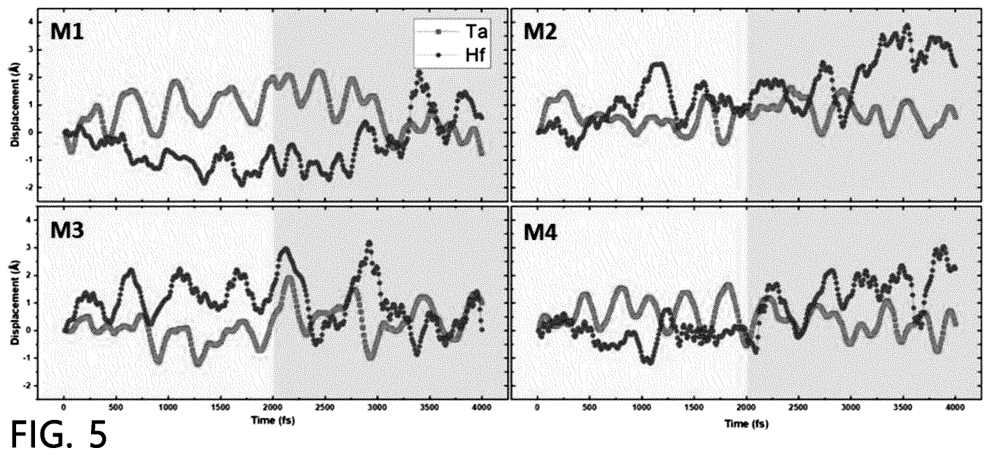

FIG. 5 illustrates the AIMD simulation results for each candidate material.

It shows the simulated changes in surface oxide composition over time for each candidate material.

Through these processes, the processor 110 can accurately and rapidly screen candidate materials suitable for use as ultra-high temperature ceramics without conducting ultra-high temperature simulation experiments.

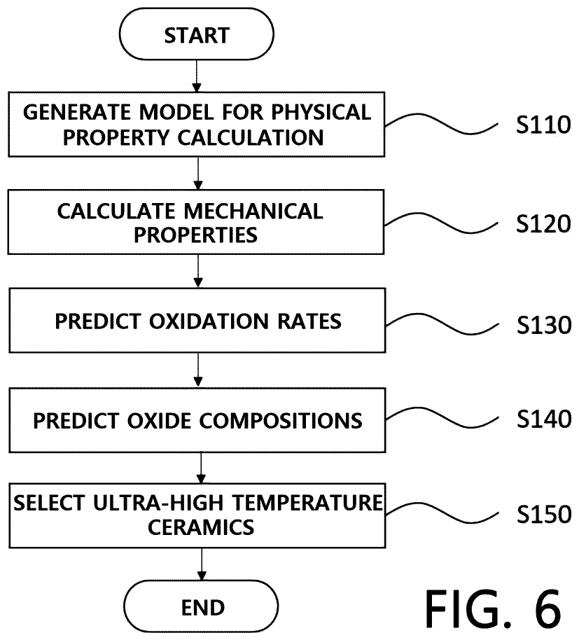

FIG. 6 is a schematic flowchart of a high throughput oxidation simulation method for selecting ultra-high temperature ceramics according to another preferred embodiment of the present disclosure.

The high throughput oxidation simulation method according to the present disclosure may be performed by a high throughput oxidation simulation apparatus including one or more processors and a memory.

To select ultra-high temperature ceramics, a model is first generated to automatically perform first-principles calculation (Ab initio) according to the type and amount of dopant elements in the candidate materials (S110).

Mechanical properties are then calculated using the generated model (S120), and candidate materials that do not meet predetermined criteria for mechanical properties are filtered out in advance, thereby saving time and resources required for subsequent oxidation simulations.

Next, the oxidation rate of the candidate materials at ultra-high temperatures is predicted (S130), and the NEB (nudged elastic band) calculation method may be used for this prediction.

Finally, the composition of oxides formed on the candidate materials at ultra-high temperatures is predicted (S140) to estimate the oxidation rate, and the AIMD (Ab initio Molecular Dynamics) simulation method may be used for this purpose.

Through these steps—mechanical property evaluation, oxidation rate prediction, and oxide composition analysis—candidate materials suitable for use as ultra-high temperature ceramics are ultimately selected (S150).

Even if these simulation results are not very accurate, they can suggest directions for composition selection and can significantly reduce actual experiments compared to the conventional method of obtaining the desired composition by repeating trial and error, which can save a lot of time and cost.

In the detailed description of the present disclosure, although specific embodiments have been described, it is apparent that various modifications are possible without departing from the scope of the present disclosure. Therefore, the scope of the present disclosure is not limited to the described embodiments and should be defined by the following claims and their equivalents.

What is claimed is:

1. A high throughput oxidation simulation apparatus for selecting ultra-high temperature ceramics, comprising:
a memory storing one or more instructions; and
a processor configured to execute the one or more instructions stored in the memory,
wherein the processor is configured to:
generate a model for calculating physical properties of candidate materials based on both a material intended for selecting ultra-high temperature ceramics and the type and amount of dopant elements,
calculate mechanical properties of the candidate materials using the generated model, predict oxidation rates of the candidate materials,
predict surface oxide compositions of the candidate materials, and
select ultra-high temperature ceramic materials based on the mechanical properties, oxidation rates, and surface oxide compositions of the candidate materials.

2. The high throughput oxidation simulation apparatus for selecting ultra-high temperature ceramics according to claim 1, wherein the processor is configured to calculate the oxidation rates of the candidate materials through NEB (nudged elastic band) calculations.

3. The high throughput oxidation simulation apparatus for selecting ultra-high temperature ceramics according to claim 1, wherein the processor is configured to calculate the surface oxide compositions and oxidation rates of the candidate materials through AIMD (Ab initio Molecular Dynamics) simulation.

4. The high throughput oxidation simulation apparatus for selecting ultra-high temperature ceramics according to claim 1, wherein the processor is configured to predict the oxidation rates and surface oxide compositions of the candidate materials only when the mechanical properties of the candidate materials meet or exceed a predetermined criterion.

5. The high throughput oxidation simulation apparatus for selecting ultra-high temperature ceramics according to claim 4,
wherein the processor is configured to calculate the synthesizability of the dopant elements for the candidate materials when the mechanical properties of the candidate materials meet or exceed a predetermined criterion,
and to predict the oxidation rates and surface oxide compositions of the candidate materials only when the dopant elements are determined to be synthesizable.

6. A high throughput oxidation simulation method performed by a high throughput oxidation simulation apparatus including one or more processors and memory, the method comprising:
generating a model for calculating physical properties of candidate materials based on both a material intended for selecting ultra-high temperature ceramics and the type and amount of dopant elements;
calculating mechanical properties of the candidate materials using the generated model;
predicting oxidation rates of the candidate materials;
predicting surface oxide compositions of the candidate materials; and
selecting ultra-high temperature ceramic materials based on the mechanical properties, oxidation rates, and surface oxide compositions of the candidate materials.

7. The high throughput oxidation simulation method for selecting ultra-high temperature ceramics according to claim 6, wherein the step of predicting oxidation rates comprises calculating the oxidation rates of the candidate materials through NEB (nudged elastic band) calculations.

8. The high throughput oxidation simulation method for selecting ultra-high temperature ceramics according to claim 6, wherein the step of predicting surface oxide compositions comprises calculating the surface oxide compositions and oxidation rates of the candidate materials through AIMD (Ab initio Molecular Dynamics) simulation.

9. The high throughput oxidation simulation method for selecting ultra-high temperature ceramics according to claim 6, wherein, after the step of calculating mechanical properties, the oxidation rates and surface oxide compositions of the candidate materials are predicted only when the mechanical properties of the candidate materials meet or exceed a predetermined criterion.

10. The high throughput oxidation simulation method for selecting ultra-high temperature ceramics according to claim 9,
further comprising, after the step of calculating mechanical properties,
calculating the synthesizability of the dopant elements for the candidate materials when the mechanical properties of the candidate materials meet or exceed a predetermined criterion.
